(19) United States
(12) Patent Application Publication
(10) Pub. No.: US 2025/0228601 A1
(43) Pub. Date: Jul. 17, 2025

(54) ENDOSCOPE TREATMENT TOOL AND TREATMENT METHOD

(71) Applicant: Olympus Corporation, Tokyo (JP)

(72) Inventors: Yasuhito SUGA, Musashino-shi (JP); Yuta MUYARI, Hino-shi (JP); Chika MIYAJIMA, Hachioji-shi (JP); Hiromasa KATO, Tokyo (JP); Takanobu YANAI, Hachioji-shi (JP)

(21) Appl. No.: 19/015,602

(22) Filed: Jan. 9, 2025

Related U.S. Application Data
(60) Provisional application No. 63/620,006, filed on Jan. 11, 2024.

(51) Int. Cl. A61B 18/08; A61B 18/00; A61B 90/00

(52) U.S. Cl. CPC A61B 18/082; A61B 90/03; A61B 2018/00083; A61B 2018/00101; A61B 2018/00589; A61B 2090/036

(57) ABSTRACT

An endoscopic treatment tool can include a sheath, a rod arranged on a distal side of the sheath, an electrode connected to a distal end of the rod, and an insulator located distally relative the electrode, wherein at least a portion of the electrode protrudes outward, in a radial direction of the rod, from an outer surface of a proximal end of the insulator.

### CROSS-REFERENCE TO RELATED APPLICATIONS

The present application claims priority based on US Patent Provisional Application No. 63/620,006 filed in the United States on Jan. 11, 2024, the contents of which are incorporated herein by reference.

### TECHNICAL FIELD

This disclosure relates to an endoscopic treatment tool and treatment method.

### BACKGROUND

In endoscopic treatments such as ESD (endoscopic submucosal dissection), as shown in PCT International Publication No. WO 2014/061701 (Patent Document 1) and the like, endoscopic treatment tools such as high-frequency knives can be used. In such treatments, a surgeon uses endoscopic treatment tools such as high-frequency knives to perform incisions of biological tissues.

### SUMMARY

When performing ESD procedures, the treatment tool must be frequently replaced depending on the purpose of the treatment. For this reason, a high-frequency knife described in Patent Document 1 and the like is required to be multifunctional so that it can easily perform tissue marking in addition to tissue incision without replacing the treatment tool.

The present disclosure provides an endoscopic treatment tool that can suitably perform tissue marking and tissue incision.

The endoscopic treatment tool according to the first aspect of the present disclosure can include a sheath, a rod arranged on a distal side of the sheath, an electrode connected to a distal end of the rod, and an insulator provided at a more distal side than a position of the electrode. A portion of the electrode can protrude outward, in a radial direction of the rod, from an outer surface of a proximal end of the insulator.

The endoscopic treatment tool disclosed herein can be used to effectively mark and incise tissue.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an example of an overall view of an endoscopic treatment system according to a first embodiment of the present disclosure.

FIG. 2 is an example of an overall view showing a treatment tool of the endoscopic treatment system.

FIG. 3 is an example of a perspective view of the distal end of the treatment tool.

FIG. 4 is an example of a side view of the distal end of the treatment tool.

FIG. 5 is an example of a side view of the distal end of the treatment tool.

FIG. 6 is an example of perspective view of the distal end of the treatment tool as viewed from the proximal end side.

FIG. 7 is an example of a front view of the distal end of the treatment tool.

FIG. 8 is an example of a view showing a marking step.

FIG. 9 is an example of a view showing an incision and peeling step.

FIG. 10 is an example of a view showing a modified insulating chip and modified electrodes of the treatment tool.

FIG. 11 an example of a front view of the modified insulating chip as viewed from the longitudinal axis direction.

FIG. 12 is an example of a view showing another modified insulating chip and another modified electrode.

FIG. 13 is an example of a view showing another modified insulating chip and another modified electrode.

FIG. 14 is an example of a view showing another modified insulating chip and another modified electrode.

FIG. 15 is an example of a cross-sectional view taken along line X1-X1 shown in FIG. 14.

FIG. 16 is an example of a cross-sectional view showing another aspect of the same modified example of the insulating chip.

FIG. 17 is an example of a diagram showing another modified example of the insulating chip and another modified example of the electrode.

FIG. 18 is an example of a cross-sectional view taken along line X2-X2 shown in FIG. 17.

FIG. 19 is an example of a cross-sectional view showing another aspect of the same modified example of the insulating chip.

FIG. 20 is an example of a side view of the distal end of a treatment tool of an endoscopic treatment system according to a second embodiment of the present disclosure.

FIG. 21 is an example of a side view of the distal end of a treatment tool of an endoscopic treatment system according to a third embodiment of the present disclosure.

### DETAILED DESCRIPTION

### First Embodiment

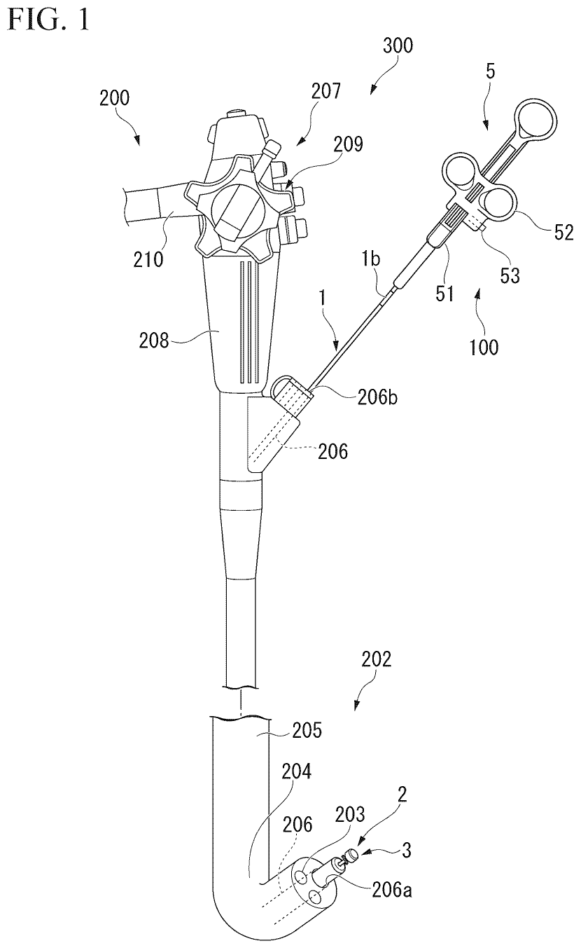

An endoscopic treatment system 300 according to a first embodiment of the present disclosure will be described with reference to FIGS. 1 to 9. FIG. 1 is an example of an overall view of the endoscopic treatment system 300 according to this embodiment.

### [Endoscope Treatment System 300]

As shown in FIG. 1, the endoscopic treatment system 300 can include an endoscope 200 and a treatment tool 100. The treatment tool 100 can be inserted into the endoscope 200 for use.

### [Endoscope 200]

The endoscope 200 can be a flexible endoscope, and can include an insertion portion 202 that is configured to be inserted into the body from the distal end, and an operating portion 207 attached to the proximal end of the insertion portion 202.

The insertion portion 202 can include an imaging portion 203, a bending portion 204, and a flexible portion 205. The imaging portion 203, the bending portion 204, and the flexible portion 205 can be arranged in this order from the distal end of the insertion portion 202. Inside the insertion portion 202, a channel 206 can be provided for inserting the treatment tool 100. A distal end opening 206a of the channel 206 can be provided at the distal end of the insertion portion 202.

The imaging portion 203 can be equipped with an imaging element such as a CCD or CMOS, or any similar imaging device capable of capturing an image of the area to be treated. The imaging portion 203 can capture an image of the distal end of the treatment tool 100 when the treatment tool 100 protrudes from the distal end opening 206a of the channel 206.

The bending portion 204 can bend in response to the operation of the operating portion 207 by a user (e.g., a surgeon). The flexible portion 205 can include a flexible tubular portion.

The operating portion 207 can be connected to the flexible portion 205. The operating portion 207 can include a grip 208, an input portion 209, a proximal end opening 206b of the channel 206, and a universal cord 210. The grip 208 can be a portion such as a handle that is held by the surgeon. The input portion 209 can accept operation input for bending the bending portion 204.

The universal cord 210 can include a video signal line that outputs the image captured by the imaging portion 203a to the outside. The video signal line can be connected to a display device such as a liquid crystal display via an image processing device equipped with a processor or the like.

### [Treatment Tool 100]

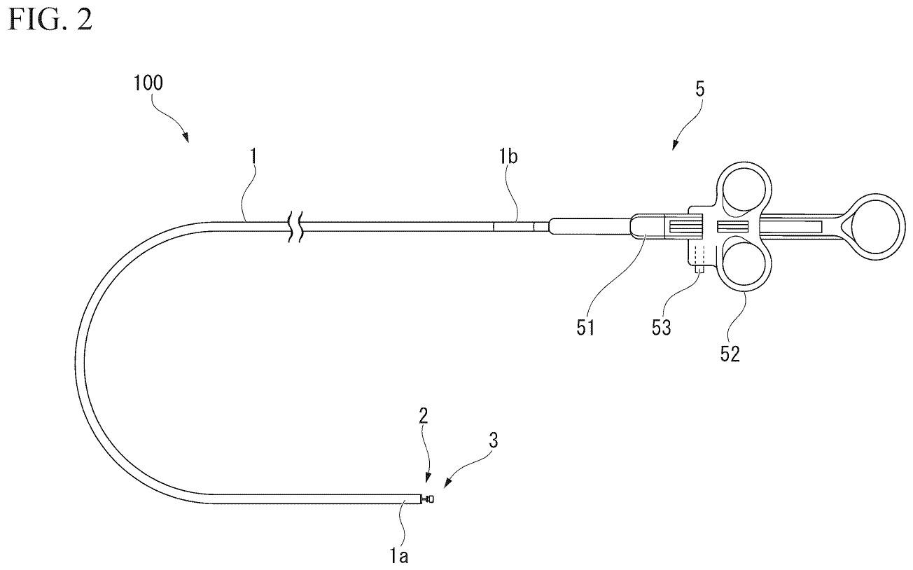

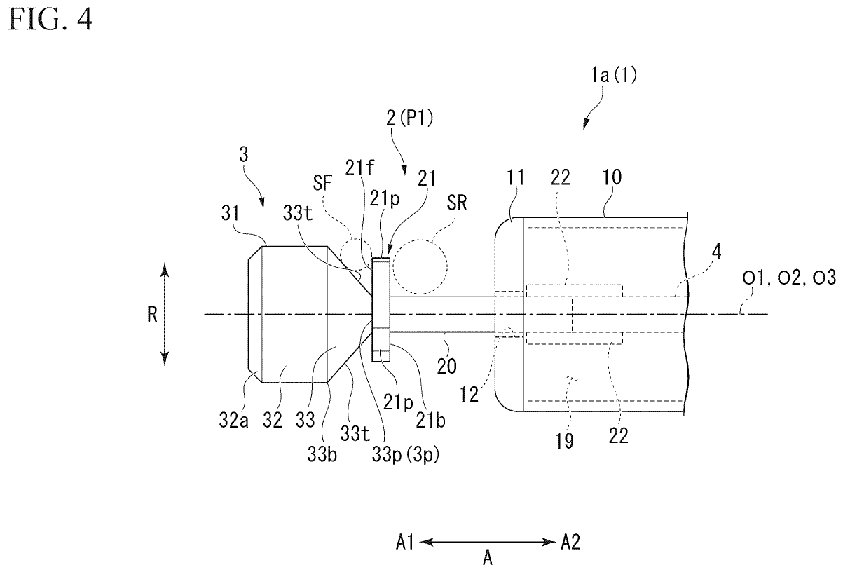

FIG. 2 is an example of an overall view showing the treatment tool 100. The treatment tool (endoscopic treatment tool, high-frequency treatment tool) 100 can be an ESD knife. The treatment tool 100 can include a sheath 1, a knife 2, an insulating chip 3, an operating wire 4 (see FIG. 4), and a control portion 5. In the following description, in the longitudinal direction (longitudinal direction, axial direction) A of the treatment tool 100, the side inserted into the patient's body is referred to as the “distal side A1” and the operating portion 5 side is referred to as the “proximal side A2”.

The sheath 1 can be a long tubular member extending from the distal end 1a to the proximal end 1b. The sheath 1 can be inserted into the channel 206 of the endoscope 200 and can advance and retract through the channel 206. As shown in FIG. 1, when the sheath 1 is inserted into the channel 206, the distal end 1a of the sheath 1 can protrude and retract from the distal end opening 206a of the channel 206.

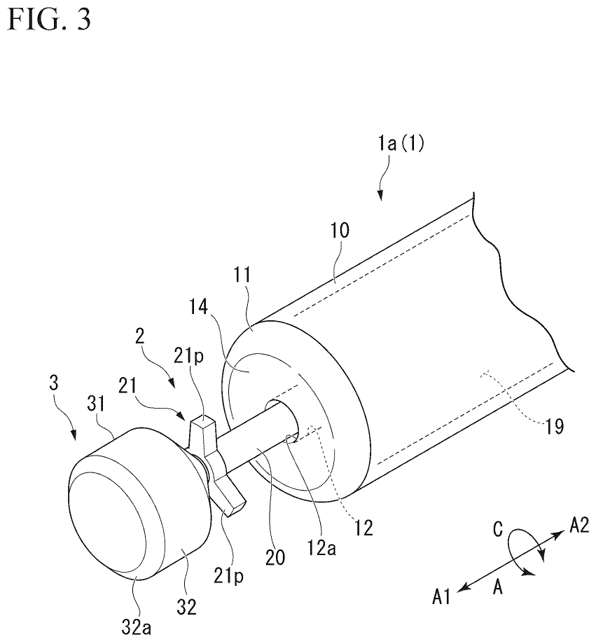

FIG. 3 is an example of a perspective view of the distal end of the treatment tool 100. The sheath 1 can include an outer tube 10 extending in the longitudinal direction A, and a distal end member 11 provided at the distal end of the outer tube 10. The sheath 1 can be formed by integrally molding the outer tube 10 and the distal end member 11.

The distal end member 11 can be formed in a cylindrical shape. Note that “cylindrical” includes shapes close to a cylindrical shape (e.g., substantially cylindrical) in addition to a strictly cylindrical shape. The distal end member 11 can be formed of an insulating material such as resin. The distal end member 11 can include a through hole 12.

The through hole 12 can be a hole provided in the distal end member 11 and can pass through the distal end member 11 in the longitudinal axis direction A. The distal end of the through hole 12 can communicate or engage with a first opening 12a formed in the distal end surface 14 of the distal end member 11. The proximal end of the through hole 12 can communicate or engage with the internal space 19 of the outer tube 10.

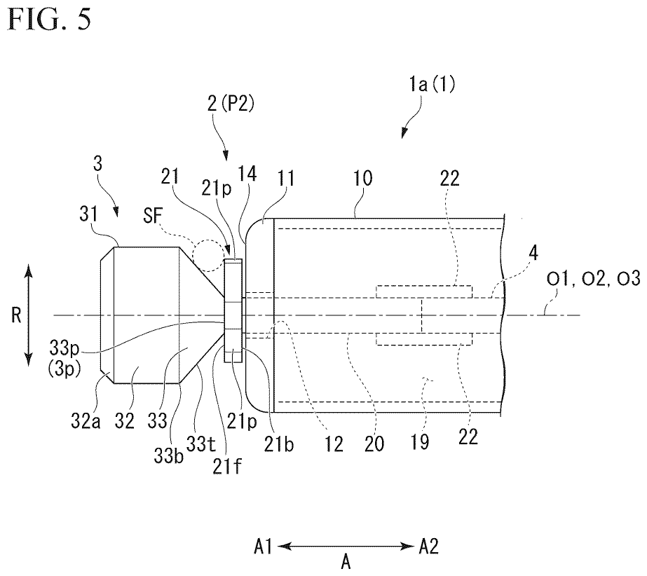

FIGS. 4 and 5 are examples of side views of the distal end of the treatment tool 100. The knife (electrode) 2 can be a metal member. The knife 2 can be formed of a material such as stainless steel. The knife 2 can be conductive and can be energized with high-frequency current. The knife 2 can include a rod 20, an electrode 21, and a connector 22.

The rod (blade, electrode body) 20 can be a metal round bar-shaped member. Note that “round bar-shaped” includes shapes close to a round bar shape (e.g., a substantially round bar shape) as well as a strict round bar shape. The rod 20 can be disposed on the distal end side A1 of the sheath 1. The operating wire 4 can be attached to the proximal end of the rod 20.

The rod 20 can be inserted through the through hole 12 of the distal end member 11 of the sheath 1 along the longitudinal axis direction A and can freely protrude and retract from the first opening 12a to the distal end side A1. The rod 20 can be be fixed in a state where it protrudes from the first opening 12a to the distal end side A1 and cannot advance or retract.

The central axis O2 of the rod 20 in the longitudinal axis direction A can coincide with the central axis O1 of the sheath 1 in the longitudinal axis direction A. Note that “coinciding” includes a state in which they coincide strictly as well as a state in which they almost coincide.

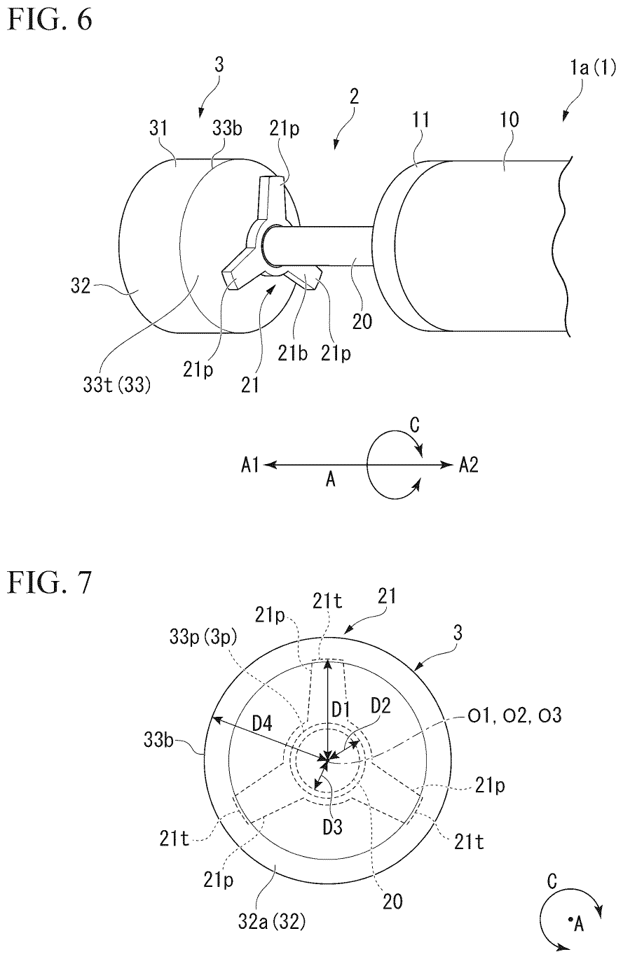

FIG. 6 is an example of a perspective view of the distal end of the treatment tool 100 as seen from the proximal end side A2. The electrode (flange, enlarged diameter portion) 21 can be connected to the distal end of the rod 20 and can be a plate-shaped conductive member extending from the outer circumferential surface of the distal end of the rod 20. The electrode 21 can include a protruding portion 21p extending in a direction intersecting the longitudinal axis of the rod 20. In this embodiment, the protruding portion 21p can protrude outward in the radial direction R of the rod 20 beyond the outer surface at the proximal end of the insulating chip (insulator) 3 described later. A planar proximal end surface (rear surface) 21b can be formed on the proximal end side A2 of the electrode 21. The proximal end surface 21b is not limited to a planar surface, and can be, for example, a surface having projections and recesses.

The connector (connecting member) 22 can be a cylindrical member made of metal. Note that “cylindrical” includes shapes close to a cylindrical shape (substantially cylindrical) as well as a strict cylindrical shape. The connector 22 can connect the rod 20 and the operating wire 4.

As shown in FIG. 4, when the knife 2 is advanced relative to the sheath 1, the distal end of the connector 22 can come into contact with the distal end member 11. The contact between the distal end of the connector 22 and the distal end member 11 can position the knife 2 at the first position P1, which is the position on the most distal side A1.

As shown in FIG. 5, when the knife 2 is retracted relative to the sheath 1, the proximal end surface 21b of the electrode 21 can come into contact with the distal end surface 14 of the distal end member 11. The contact between the proximal end surface 21b of the electrode 21 and the distal end member 11 can position the knife 2 at the second position P2, which is the position on the most proximal side A2.

The knife 2 can be advanced and retracted between the second position P2 and the first position P1 by the operating wire 4 advancing and retracting. The knife 2 can be fixed so as not to be advanced and retracted in a state where it protrudes from the first opening 12a to the distal side A1.

As shown in FIG. 4, by moving the knife 2 closer to the first position P1, a rear space SR can be secured on the proximal side A2 of the electrode 21. By securing the rear space SR of the electrode 21, the surgeon can suitably perform an incision procedure using the electrode 21.

A high-frequency current can be supplied to the knife 2 from the operating wire 4 connected to the operating portion 5. When a high-frequency current is supplied to the knife 2 from the operating wire 4, the rod 20 and the electrode 21 can function as a monopolar electrode that outputs a high-frequency current to the biological tissue.

The insulating chip (insulator) 3 can be formed of an insulating material such as ceramic or resin. The insulating chip 3 can be provided on the distal side A1 of the electrode 21. In this embodiment, the proximal end 3p of the insulating chip 3 can be fixed to the rod 20 in contact with the electrode 21. At least the protruding portion 21p of the electrode 21 can protrude outward in the radial direction R from the outer surface (the proximal end portion 33p of the tapered surface 33t described later) at the proximal end 3p of the insulating chip 3.

The central axis O3 in the longitudinal axis direction A of the insulating chip 3 can coincides with the central axis O1 in the longitudinal axis direction A of the sheath 1. Note that “coincidence” includes a state in which the central axis O3 and the sheath 1 almost coincide or substantially coincide with each other in addition to a state in which the central axis O1 and the sheath 1 coincide with each other strictly.

The insulating chip 3 can include a distal end portion 32 arranged on the distal side A1 and a proximal end portion 33 arranged on the proximal side A2. The distal end portion 32 and the proximal end portion 33 can be arranged and connected in the longitudinal axis direction A. The distal end portion 32 and the proximal end portion 33 can be formed integrally or can be formed by connecting separate members.

The distal end portion 32 can be formed in a cylindrical shape and can include a chamfered portion 32a on the outer periphery of the distal end portion. The central axis of the distal end portion 32 in the longitudinal axis direction A can coincide with the central axis O3.

The proximal end portion 33 can be formed in a cone shape. Therefore, in such an example, the outer diameter of the proximal end 3p (proximal end portion 33p of the proximal end portion 33) of the insulating chip 3 is smaller than the outer diameter of the distal end of the insulating chip (distal end of the distal end portion 32). The central axis of the proximal end portion 33 in the longitudinal axis direction A can coincide with the central axis O3. The proximal end portion 33 can have a tapered surface (inclined surface, inclined portion) 33t that reduces in diameter toward the electrode 21 (toward the proximal end side A2). Therefore, in this example, at least the protruding portion 21p of the electrode 21 is not covered by the insulating chip 3, and the tapered surface 33t is separated from a part of the electrode 21 with a gap in the longitudinal axis direction A of the rod 20. A part of the electrode 21 (protruding portion 21p) can protrude outward in the radial direction R of the rod 20 beyond the outer surface at the proximal end 3p of the insulating chip 3. In the longitudinal axis direction A of the rod 20, the tapered surface 33t and the protruding portion 21p can be spaced apart with a gap, and are disposed so that the tapered surface 33t and the distal end surface 21f of the protruding portion 21p face each other.

In this embodiment, the proximal end 3p of the tapered surface 33t (proximal end of the proximal end portion 33, proximal end of the insulating chip 3) and the base of the electrode 21 are in contact. That is, the radially outer side of the electrode 21 is separated from the insulating chip 3, and the radially inner side is in contact with the insulating chip 3. However, the electrode 21 and the insulating chip 3 do not necessarily need to be in contact. The proximal end 3p of the tapered surface 33t (proximal end portion 33p of the proximal end portion 33) and a part of the base of the electrode 21 can be separated with a small gap.

The distance from the electrode 21 can increase from the proximal end portion 33p of the tapered surface 33t toward the distal end. Therefore, a front space (gap) SF is formed between the tapered surface 33t and the protruding portion 21p. That is, the insulating chip 3 and the electrode 21 can be fixed to the rod 20 so that the tapered surface 33t and the protruding portion 21p can be separated by a gap in the longitudinal axis direction A of the rod 20, and the tapered surface 33t and the distal end surface 21f of the protruding portion 21p face each other. The rod 20 and the electrode 21 can be molded as one piece. The surgeon can perform incision procedures, marking, etc. using the electrode 21 by securing a space (gap) SF in front of the protruding portion 21p.

FIG. 7 is an example of a front view of the distal end of the treatment tool 100. The electrode 21 can include three protruding portions 21p. The three protruding portions 21p can be arranged at equal intervals along the circumferential direction C relative to the longitudinal axis direction A. When viewed from the front in a direction along the longitudinal axis direction A, the electrode 21 is formed in a trifurcated shape. The electrode 21 can be formed in a quadruple or quintuple shape. That is, the electrode 21 can be formed in a radial shape extending radially outward from the central axis O2 of the rod 20 in the radial direction R. The electrode 21 can also be formed in a disk shape or a polygonal shape.

In a direction perpendicular to the longitudinal axis of the rod 20, the shortest distance D2 in the radial direction R from the central axis O2 of the rod 20 to the proximal end 3p of the outer surface 31 of the insulating chip 3 (proximal end portion 33p of the tapered surface 33t, proximal end portion 33p of the outer peripheral surface of the proximal end portion 33) can be smaller than the distance D1 in the radial direction R from the central axis O2 of the rod 20 to the top 21t of the protruding portion 21p of the electrode 21, and can be larger than the radius D3 of the rod 20 (D1>D2>D3). In addition, the shortest distance D4 from the central axis O2 of the rod 20 to the distal end (distal end of the outer peripheral surface of the proximal end portion 33) 33b of the tapered surface 33t formed on the proximal end portion 33 of the insulating chip 3 can be greater than the distance D1 from the central axis O2 of the rod 20 to the apex 21t of the protruding portion 21p of the electrode 21 (D4>D1). The distal end (apex) 21t of the protruding portion 21p can be disposed radially outward of the proximal end 3p of the outer surface 31 of the insulating chip 3.

The operating wire 4 can be a metal wire that passes through the internal space (pipe, lumen) 19 of the outer tube 10. The operating wire 4 can be formed of a material such as stainless steel. The distal end of the operating wire 4 can be connected to the rod 20, and the proximal end of the operating wire 4 can be connected to the operating portion 5. The operating wire 4 can be a hollow wire. In this case, by providing an opening at the distal end of the insulating chip 3 and communicating the opening with the internal space of the operating wire 4, a fluid such as physiological saline can be discharged from the distal end of the insulating chip 3.

As shown in FIGS. 1 and 2, the operating portion 5 can include an operating portion main body 51, a slider 52, and a power supply connector 53.

The distal end of the operating portion main body 51 can be connected to the proximal end 1b of the sheath 1. The operating portion main body 51 can include an internal space through which the operating wire 4 can be inserted. The operating wire 4 can pass through the internal space 19 of the outer tube 10 and the internal space of the operating portion main body 51 and can extend to the slider 52.

The slider 52 can be attached to the operating portion main body 51 so as to be movable along the longitudinal axis direction A. The proximal end of the operating wire 4 can be attached to the slider 52. The surgeon can advance and retract the slider 52 relative to the operating portion body 51, thereby advancing and retracting the operating wire 4, the knife 2, and the insulating chip 3.

The power supply connector 53 can be fixed to the slider 52. The power supply connector 53 can be connected to a high-frequency power supply device and can be connected to the proximal end of the operating wire 4 via a conductive wire. The power supply connector 53 can supply high-frequency current supplied from the high-frequency power supply device to the rod 20 via the operating wire 4. The power supply connector 53 can be fixed to the operating portion body 51, not to the slider 52.

### [Method of Using the Endoscopic Treatment System 300]

Next, a procedure (method of using the endoscopic treatment system 300) using the endoscopic treatment system 300 of this embodiment will be described. Specifically, an incision and ablation treatment of a lesion in an endoscopic treatment such as ESD (endoscopic submucosal dissection) will be described.

As a preparatory step, the surgeon can identify the lesion using any appropriate method. For example, the surgeon can insert the insertion portion 202 of the endoscope 200 into the digestive tract (e.g., esophagus, stomach, duodenum, large intestine) and identify the lesion while observing the image obtained by the imaging portion 203 of the endoscope.

### <Insertion Step>

The surgeon can insert the treatment tool 100 into the channel 206 and protrude the distal end 1a of the sheath 1 from the distal end opening 206a of the insertion portion 202. The surgeon can advance the slider 52 of the operating portion 5 relative to the operating portion body 51 and protrude the knife 2 and the insulating chip 3.

### <Marking Step>

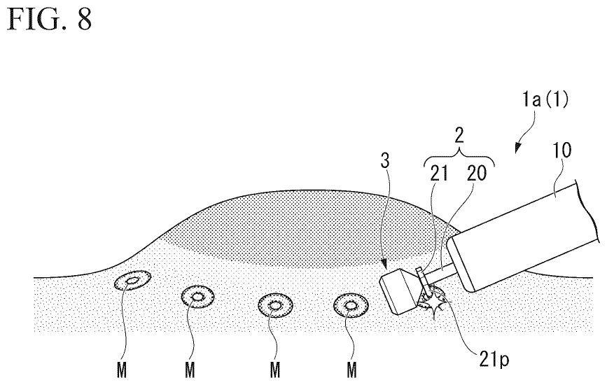

FIG. 8 is a diagram showing the marking step. The surgeon can advance the slider 52 of the operating portion 5 relative to the operating portion body 51 and position the knife 2 on the distal side A1. That is, the knife 2 can be positioned at the first position P1, which is the position of the distal side A1. The surgeon can cauterize and coagulate the biological tissue (mucosal surface) around the lesion by passing a current through the protruding portion 21p of the electrode 21 while it is in contact with the mucosal surface. As a result, marking M can be applied to the mucosal surface. At this time, by passing a current through the electrode 21 and the outer surface 31 of the insulating chip 3 while they are in contact with the mucosal surface, it is possible to prevent the electrode 21 from being deeply embedded in the tissue and passing a current through the biological tissue against the surgeon's intention. Since the protruding portion 21p is separated from the insulator 3 by a gap, the surgeon can use the distal end of the protruding portion 21p to suitably apply marking M to the biological tissue. The marking M on the biological tissue is different from incision in that the tissue is denatured by coagulating the biological tissue (mucosal surface).

### <Incision and Dissection Step>

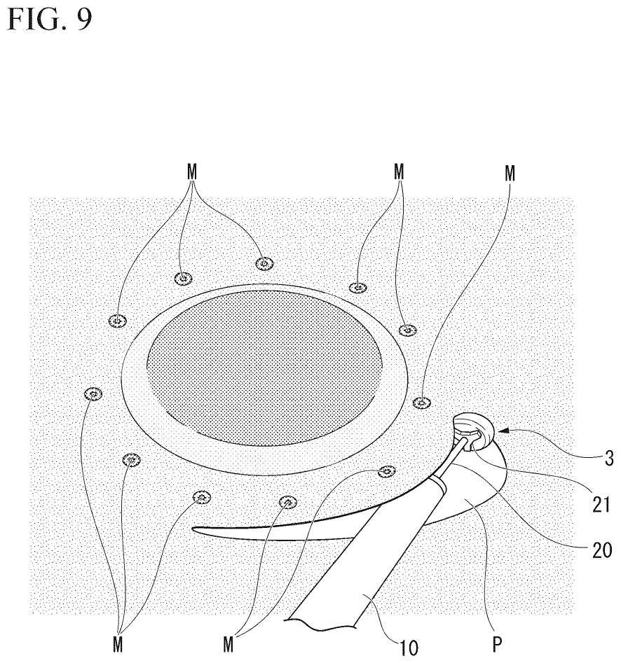

FIG. 9 is an example of a diagram showing the incision and dissection step. The surgeon can perform the incision and dissection procedure. As shown in FIG. 9, the surgeon can create a precut (an entry point for starting the incision) P in the mucosal layer and/or submucosal layer by passing electricity through the electrode 21 and rod 20 with the insulating chip 3 and electrode 21 inserted under the mucosal layer. The surgeon can incise the mucosa of the lesion by moving the electrode 21 from the precut P while high-frequency current is being passed through it. As shown in FIG. 9, the surgeon can peel off the submucosal layer of the incised lesion while lifting the mucosa of the incised lesion to expose the submucosal layer while passing high-frequency current through it.

The surgeon can continue the above-mentioned operation (treatment) as necessary, and finally resect the lesion, completing the ESD procedure.

The treatment tool 100 according to this embodiment can be used to suitably mark and incise the biological tissue.

The first embodiment of the present disclosure has been described above in detail with reference to the drawings, but the specific configuration is not limited to this embodiment, and design modifications and the like are also included within the scope of the gist of the present disclosure. In addition, the components shown in the above- mentioned embodiments and modifications can be appropriately combined to configure the present disclosure.

### Modification 1

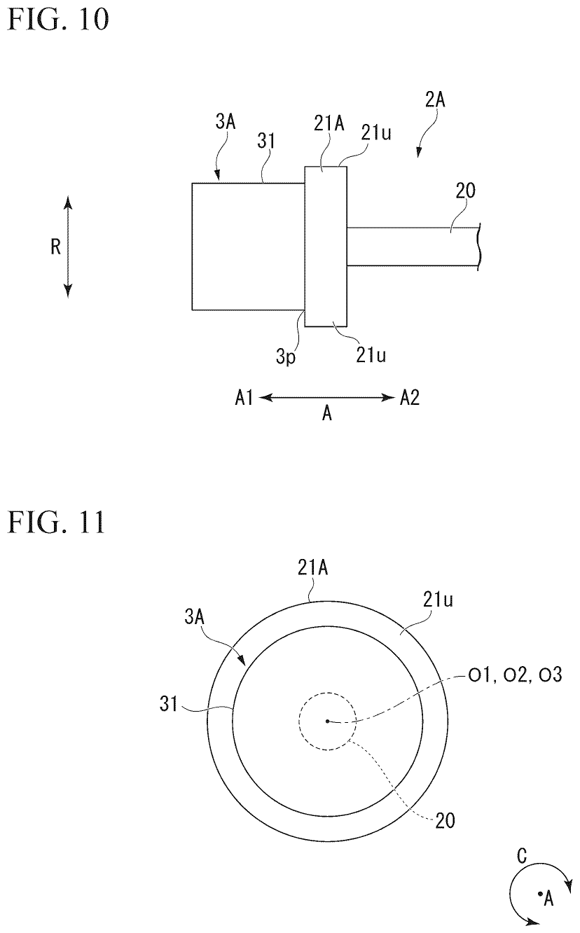

FIG. 10 shows an example of insulating chip 3A, which is a modification of the insulating chip 3, and an electrode 21A, which is a modification of the electrode 21. FIG. 11 is an example of a front view of the insulating chip 3A as viewed from a direction along the longitudinal axis A.

The insulating chip 3 of the first embodiment can include a tapered surface 33t formed on the proximal end portion 33. However, the insulating chip 3 does not necessarily have to have a tapered surface. For example, the insulating chip 3A can be formed in a cylindrical shape from the distal end to the proximal end. The proximal end 3p of the insulating chip 3A can be fixed to the rod 20 in a state of contact with the electrode 21A. The electrode 21A can be a disc-shaped conductive member provided at the distal end of the rod 20. As shown in FIG. 11, the outer periphery (protrusion 21u) of the electrode 21A can be disposed radially outward from the outer surface 31 of the insulating chip 3A over the entire circumference. The electrode 21A can be formed in a radial shape such as a trifurcated shape, a disk shape, or a polygonal shape. Even in this case, at least a part of the outer periphery (protrusion 21u) of the electrode 21A can be disposed radially outward from the outer surface 31 of the insulating chip 3A.

### Modification 2

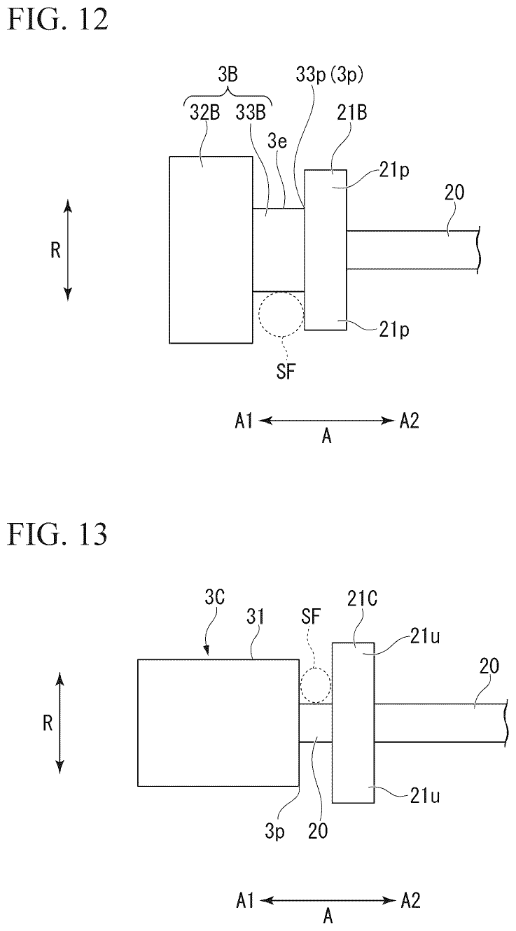

FIG. 12 shows an example of insulating chip 3B, which is a variation of the insulating chip 3, and an electrode 21B, which is a variation of the electrode 21. The insulating chip 3B can include a distal end portion 32B disposed on the distal side A1 and a proximal end portion 33B disposed on the proximal side A2. The distal end portion 32B and the proximal end portion 33B can be arranged and connected in the longitudinal axis direction A. The distal end portion 32B and the proximal end portion 33B can be formed integrally or can be formed by connecting separate members. The distal end portion 32B and the proximal end portion 33B can be formed in a cylindrical shape. The outer diameter of the distal end portion 32B can be larger than the outer diameter of the proximal end portion 33B. The proximal end 3p of the proximal end portion 33B of the insulating chip 3B is fixed to the rod 20 in a state of contacting the base of the electrode 21B. The electrode 21B can include a protruding portion (protrusion) 21p that protrudes outward in the radial direction R from the outer surface 3e of the proximal end 3p (the proximal end portion 33p of the proximal end portion 33B) of the insulating chip 3B. A front space (e.g., a gap) SF can be formed between the distal end portion 32B and the protruding portion 21p. When the electrode 21B is, for example, disc-shaped, the outer periphery of the electrode 21B can be disposed outward in the radial direction R from the outer surface of the proximal end portion 33B of the insulating chip 3B over the entire circumference. When the electrode 21B has a radial shape such as a trifurcated or quadrupled shape, a part of the electrode 21B (including the apex) can be disposed on the outer side of the outer surface 3e of the proximal end portion 33B of the insulating chip 3B in the radial direction R. In the longitudinal axis direction A of the rod 20, the distal end portion 32B and the protruding portion 21p can be disposed so as to be separated with a gap, and the proximal end surface of the distal end portion 32B and the distal end surface of the protruding portion 21p face each other.

### Modification 3

FIG. 13 shows an example of an insulating chip 3C, which is a modification of the insulating chip 3, and an electrode 21C, which is a modification of the electrode 21. The insulating chip 3C can be formed in a cylindrical shape. The insulating chip 3C can be fixed to the rod 20. The proximal end 3p of the insulating chip 3C is not in contact with the electrode 21C. In other words, the insulating chip 3C can be fixed to the rod 20 with the proximal end 3p of the insulating chip 3C separated from the electrode 21C with a gap. The proximal end 3p of the insulating chip 3C and the electrode 21C (including the protruding portion 21u) can be separated, and a front space (e.g., a gap) SF can be formed between the insulating chip 3C and the electrode 21C (including the protruding portion 21u). When the electrode 21C is, for example, disc-shaped, the outer periphery (protruding portion 21u) of the electrode 21C can be positioned radially outward of at least the proximal end 3p of the outer surface 31 of the insulating chip 3C over the entire circumference. In this modification, since the insulating chip 3C is cylindrical, the electrode 21C (protrusion 21u) can be disposed radially outward from the distal end of the outer surface 31 of the insulating chip 3C over the entire circumference. If the electrode 21C has a radial shape such as a trifurcated or quadruple shape, a part of the electrode 21C (protrusion 21u) can be disposed radially outward from at least the proximal end 3p of the outer surface 31 of the insulating chip 3C. In this modification, since the insulating chip 3C is cylindrical, a part of the electrode 21C (protrusion 21u) can be disposed radially outward from the distal end of the outer surface 31 of the insulating chip 3C in a front view from the direction along the longitudinal axis A.

### Modification 4

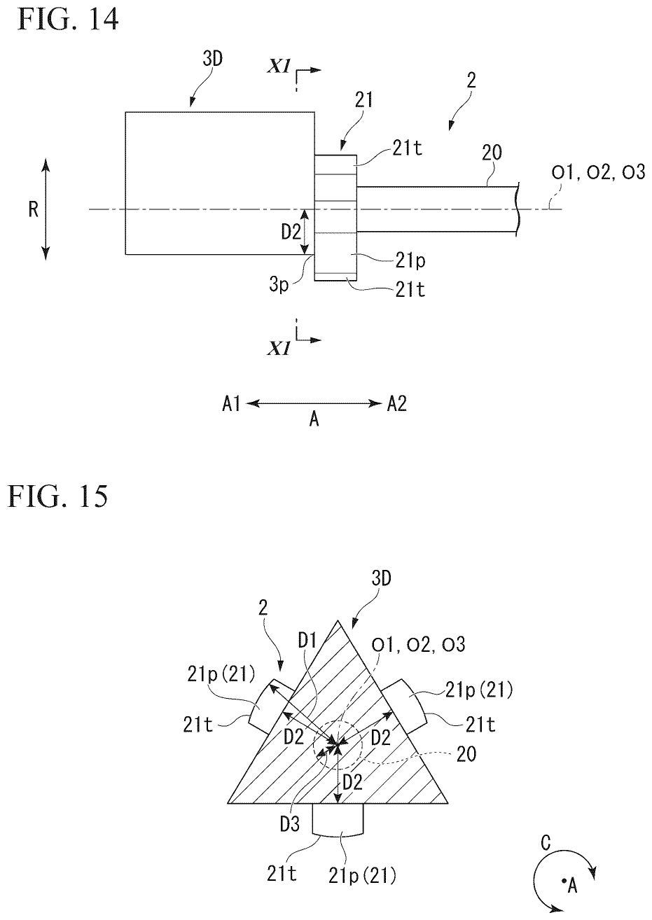

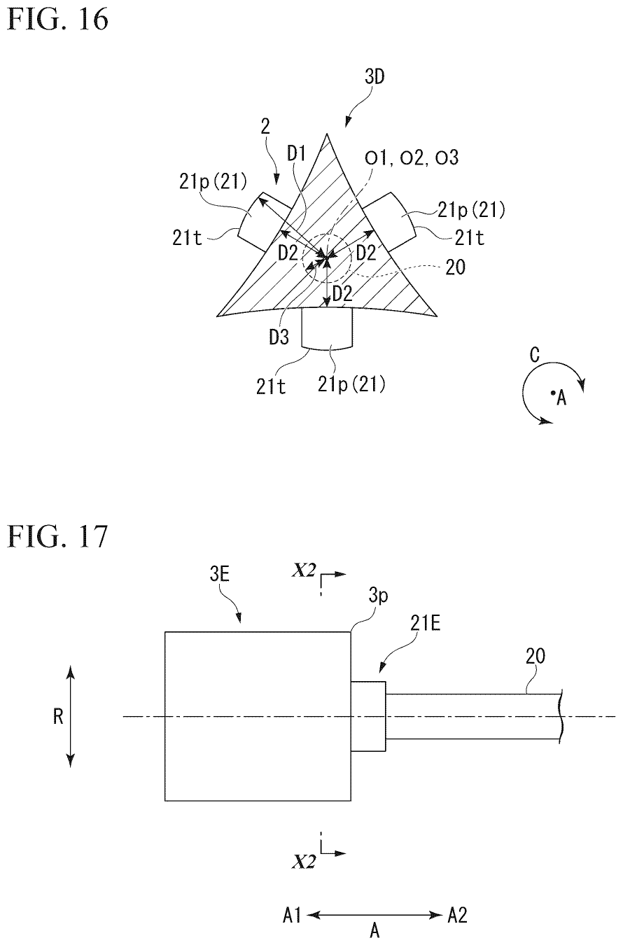

FIG. 14 is an example of a diagram showing an insulating chip 3D, which is a modification of the insulating chip 3. FIG. 15 is an example of a cross-sectional view taken along the X1-X1 line shown in FIG. 14. FIG. 16 is an example of a cross-sectional view showing another aspect of the insulating chip 3D. The electrode 21 can be formed in a trifurcated shape. At least at the proximal end of the insulating chip 3D, a cross portion perpendicular to the central axis of the rod 20 can be formed in a triangular shape. In a direction perpendicular to the longitudinal axis of the rod 20, the protruding portion 21p of the electrode 21 can protrude from the direction in which the shortest distance D2 from the central axis O2 of the rod 20 to the proximal end 3p of the outer surface of the insulating chip 3D is the smallest. That is, the direction (first direction) in which the distance from the central axis O2 of the rod 20 to the outer surface of the proximal end 3p of the insulating chip 3D can be the smallest in the radial direction R of the rod 20 and the direction (second direction) in which the electrode 21 extends in the radial direction R coincide with each other in the circumferential direction C of the rod 20. In a direction perpendicular to the longitudinal axis of the rod 20, the shortest distance D2 in the radial direction R from the central axis O2 of the rod 20 to the proximal end 3p of the outer surface of the insulating chip 3D can be smaller than the distance D1 in the radial direction R from the central axis O2 of the rod 20 to the top 21t of the protruding portion 21p of the electrode 21 and can be larger than the radius D3 of the rod 20 (D1>D2>D3). As shown in FIG. 16, the insulating chip 3D is triangular in front view from the direction along the longitudinal axis A, and each side may be curved to approach the central axis 03.

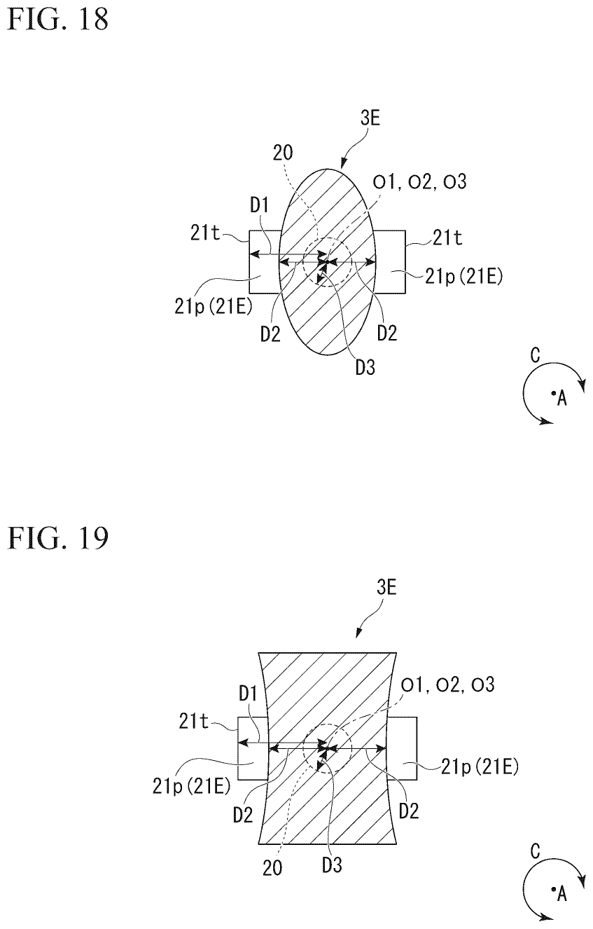

FIG. 17 shows an example of an insulating chip 3E, which is a modified example of the insulating chip 3, and an electrode 21E, which is a modified example of the electrode 21. FIG. 18 is an example of a cross-sectional view taken along the line X2-X2 shown in FIG. 17. FIG. 19 is an example of a cross-sectional view showing another aspect of the insulating chip 3E. The electrode 21E can be formed in a bifurcated shape. At least at the proximal end of the insulating chip 3E, the cross-sectional shape perpendicular to the central axis of the rod 20 is formed in an elliptical or oval shape. In the direction perpendicular to the longitudinal axis of the rod 20, the protruding portion 21p of the electrode 21E can protrude from the direction in which the shortest distance D2 from the central axis O2 of the rod 20 to the proximal end 3p of the outer surface of the insulating chip 3E is the smallest. That is, the direction in which the distance from the central axis O2 of the rod 20 to the outer surface of the proximal end 3p of the insulating chip 3E is the smallest in the radial direction R of the rod 20 and the direction in which the electrode 21E can extend in the radial direction R coincide with the circumferential direction of the rod 20. In a direction perpendicular to the longitudinal axis of the rod 20, the shortest distance D2 in the radial direction R from the central axis O2 of the rod 20 to the proximal end 3p of the outer surface of the insulating chip 3E can be smaller than the distance D1 in the radial direction R from the central axis O2 of the rod 20 to the top 21t of the protruding portion 21p of the electrode 21, and can be larger than the radius D3 of the rod 20 (D1>D2>D3). As shown in FIG. 19, the insulating chip 3E can be formed in a rectangular shape when viewed from the front in the direction along the longitudinal axis A, and the long side may be curved so as to approach the central axis O3.

### Second Embodiment

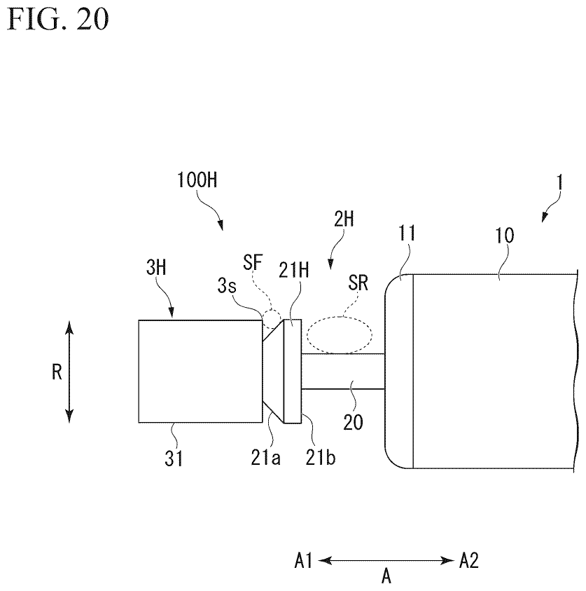

The treatment tool 100H according to the second embodiment of the present disclosure will be described with reference to FIG. 20. In the following description, components common to those already described are given the same reference numerals and redundant description will be omitted.

FIG. 20 is an example of a side view of the distal end of the treatment tool 100H. The treatment tool (endoscopic treatment tool, high-frequency treatment tool) 100H can be an ESD knife. The treatment tool 100H can include a sheath 1, a knife 2H, an insulating chip 3H, an operating wire 4, and an operating portion 5.

The knife (electrode) 2H can be a metal member. The knife 2H can be formed of a material such as stainless steel. The knife 2H can be conductive and a high-frequency current can be passed through it. The knife 2H can include a rod 20, an electrode 21H, and a connector 22.

The electrode (flange, enlarged diameter portion) 21H can be a cone-shaped conductive member provided at the distal end of the rod 20. A tapered surface 21a that narrows toward the insulating chip 3H (toward the distal side A1) can be formed on the distal side A1 of the electrode 21H. A flat proximal end surface (rear surface) 21b can be formed on the proximal side A2 of the electrode 21H. The distal end of the tapered surface 21a can be located inward in the radial direction R of the rod 20 from the proximal end of the outer surface 31 of the insulating chip 3H. In the longitudinal axis direction A of the rod 20, the tapered surface 21a and a part of the proximal end surface 3s of the insulating chip 3H can be separated by a gap, and the tapered surface 21a and a part of the proximal end surface 3s of the insulating chip 3H can be disposed so as to face each other. When the distal side A1 of the electrode 21H is conical, the proximal end of the tapered surface 21a can include an outer diameter that is the same as or slightly smaller than the outer diameter of the outer surface 31 of the insulating chip 3H. The electrode 21H is not limited to a cone shape and can be formed in a different shape, for example, a radial shape such as a trifurcated or quadrupled shape. In this case, the above- mentioned tapered surface 21a can be formed on the distal side A1 of the electrode 21H.

The insulating chip 3H can be formed in a cylindrical shape. The proximal end of the insulating chip 3H can be fixed to the rod 20 while in contact with the electrode 21H.

The treatment tool 100H according to this embodiment can suitably mark and incise the biological tissue. A front space (e.g., a gap) SF can be formed between the proximal end of the insulating chip 3H and the tapered surface 21a of the electrode 21H. By securing the front space (gap) SF of the electrode 21H, the surgeon can suitably perform incision treatment using the electrode 21H.

The second embodiment of the present disclosure has been described above in detail with reference to the drawings, but the specific configuration is not limited to this embodiment, and design changes and the like within the scope of the present disclosure are also included. In addition, the components shown in the above-mentioned embodiments and modified examples can be configured by appropriately combining them.

### Third Embodiment

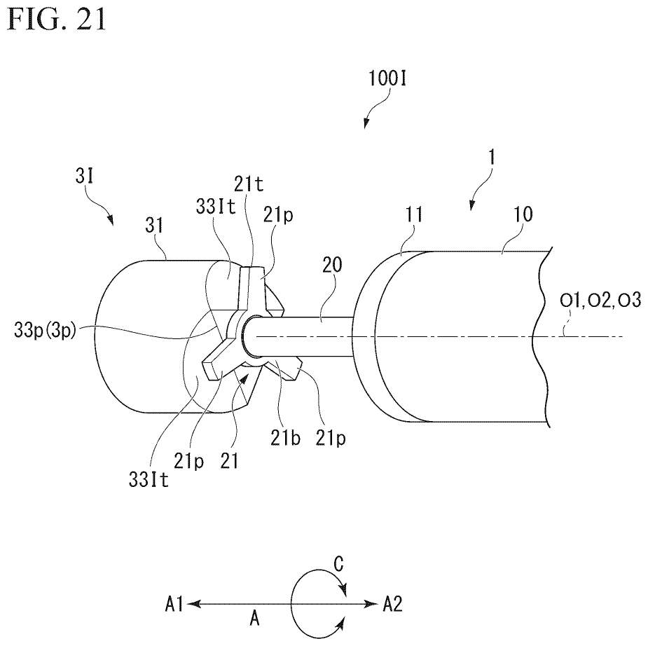

A treatment tool 1001 according to a third embodiment of the present disclosure will be described with reference to FIG. 21. In the following description, the same reference numerals will be used to designate components that are common to those already described, and duplicated descriptions will be omitted.

FIG. 21 is an example of a side view of the distal end of the treatment tool 100I. The treatment tool (endoscopic treatment tool, high-frequency treatment tool) 100I can be an ESD knife. The treatment tool 100I can include a sheath 1, a knife 2, an insulating chip 3I, an operating wire 4, and an operating portion 5.

The insulating chip 3I can be formed in a cylindrical shape. Three tapered surfaces 33It can be formed on at least a portion of the proximal end of the insulating chip 3I. The tapered surface 33It can be an inclined surface (e.g., an inclined portion) formed in a flat shape, and the normal line can face the proximal end side A2. The three tapered surfaces 33It can be evenly arranged along the circumferential direction C. In the longitudinal axis direction A, at least a portion of the tapered surface 33It can be arranged at a position facing the protruding portion 21p. Therefore, in the radial direction R of the rod 20, the direction in which the tapered surface 33It extends and the direction in which the protruding portion 21p extends can coincide with each other in the circumferential direction C of the rod 20. The proximal end of the insulating chip 3I can be fixed to the rod 20 in a state of contact with the base of the electrode 21.

At least the protruding portion 21p of the electrode 21 can protrude outward in the radial direction R from the outer surface of the proximal end 3p of the insulating chip 3I (the proximal end portion 33p of the tapered surface 33It). In the direction perpendicular to the longitudinal axis of the rod 20, the electrode 21 can protrude outward in the radial direction R in the direction in which the shortest distance from the central axis O2 of the rod 20 to the outer surface 31 of the insulating chip 3I is the shortest. That is, the direction in the radial direction R of the rod 20 in which the distance from the central axis O2 of the rod 20 to the outer surface of the proximal end 3p of the insulating chip 3I is the shortest can coincide with the direction in which the electrode 21 extends in the radial direction R in the circumferential direction C of the rod 20. In addition, the shortest distance in the radial direction R from the central axis O2 of the rod 20 to the outer surface at the proximal end 3p of the insulating chip 3I can be smaller than the distance in the radial direction R from the central axis O2 of the rod 20 to the apex 21t of the protruding portion 21p of the electrode 21 and can be larger than the radius of the rod 20.

The treatment tool 100I according to this embodiment can suitably perform marking and incision of biological tissue. Since the insulating chip 3I has a tapered surface 33It, a forward space SF can be secured at the distal side A1 of the protrusion 21p. By securing the forward space SF of the protrusion 21p, the surgeon can perform an incision procedure using the electrode 21.

The third embodiment of the present disclosure has been described above in detail with reference to the drawings, but the specific configuration is not limited to this embodiment and includes design changes and the like that do not deviate from the gist of the present disclosure. In addition, the components shown in the above-mentioned embodiment and modified examples can be appropriately combined to configure the present disclosure.

The present disclosure can be applied to endoscopic treatment tools.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

What is claimed is:

1. An endoscopic treatment tool comprising:
a sheath;
a rod located on a distal side of the sheath;
an electrode connected to a distal end of the rod; and
an insulator located distally relative to the electrode, wherein at least a portion of the electrode protrudes outward from an outer surface of a proximal end of the insulator in a radial direction of the rod.

2. The endoscopic treatment tool according to claim 1, wherein the insulator is fixed to the rod, and wherein the proximal end of the insulator is in contact with the electrode.

3. The endoscopic treatment tool according to claim 1, wherein the insulator is fixed to the rod, and wherein the proximal end of the insulator is spaced apart from the electrode.

4. The endoscopic treatment tool according to claim 1, wherein the electrode has a protrusion protruding from an outer surface at the proximal end of the insulator in a direction intersecting a longitudinal axis of the rod, wherein a shortest distance in the radial direction between a central axis of the rod and the outer surface at the proximal end of the insulator is smaller than a distance in the radial direction between the central axis of the rod and a top of the protrusion of the electrode, and wherein the shortest distance is larger than a radius of the rod.

5. The endoscopic treatment tool according to claim 1, wherein the insulator has a tapered surface, wherein the tapered surface narrows toward the electrode, and wherein he tapered surface is spaced apart from the at least a portion of the electrode that protrudes outward from the outer surface of the proximal end of the insulator in a longitudinal direction of the rod.

6. The endoscopic treatment tool according to claim 1, wherein an outer diameter of the proximal end of the insulator is smaller than an outer diameter of a distal end of the insulator.

7. The endoscopic treatment tool according to claim 1, wherein an outer periphery of the electrode protrudes outward from at least a proximal end of the outer surface of the insulator in the radial direction of the rod.

8. The endoscopic treatment tool according to claim 1, wherein a first direction in which a distance between a central axis of the rod and the outer surface of the insulator is smallest coincides with a second direction in which the electrode extends in the radial direction.

9. The endoscopic treatment tool according to claim 1, wherein the electrode has a plurality of protrusions, and wherein the plurality of protrusions extends radially outward from a central axis of the rod.

10. The endoscopic treatment tool according to claim 1, wherein the insulator has an inclined portion being a part of the proximal end of the insulator, and wherein a first direction in which the inclined portion extends from a central axis of the rod coincides with a second direction in which the electrode extends in the radial direction.

11. An endoscope treatment tool, comprising:
a sheath;
a rod disposed on a distal side of the sheath;
an electrode connected to a distal end of the rod; and
an insulator disposed distally relative to the electrode, wherein the electrode is spaced apart from a proximal end of the insulator at a radial outside of the rod, and wherein the electrode is in contact with the proximal end of the insulator at a radial inside of the rod.

12. The endoscope treatment tool according to claim 11, wherein the insulator is fixed to the rod, the proximal end of the insulator is in contact with the electrode.

13. The endoscope treatment tool according to claim 11, wherein the electrode has a protrusion protruding from an outer surface of the proximal end of the insulator in a direction intersecting a longitudinal axis of the rod.

14. The endoscope treatment tool according to claim 13, wherein a shortest distance in a radial direction between a central axis of the rod and an outer surface of the proximal end of the insulator is smaller than a distance in the radial direction between the central axis of the rod and a top of the protrusion of the electrode, and wherein the shortest distance is larger than a radius of the rod.

15. The endoscope treatment tool according to claim 13, wherein the proximal end of the insulator has a tapered surface, wherein the tapered surface narrows toward the electrode, and wherein the tapered surface is spaced apart from the protrusion in a longitudinal direction of the rod.

16. The endoscope treatment tool according to claim 11, wherein an outer diameter of the proximal end of the insulator is smaller than an outer diameter of a distal end of the insulator.

17. The endoscope treatment tool according to claim 11, wherein the electrode protrudes outward, in a radial direction of the rod, from a position where a shortest distance between a central axis of the rod and an outer surface of the insulator is shortest, in a longitudinal direction of the rod.

18. The endoscope treatment tool according to claim 11, wherein a distal end of the electrode has a tapered surface, and wherein the tapered surface narrows toward the insulator.

19. A treatment method using an endoscopic treatment tool, comprising:
abutting an electrode of the endoscopic treatment tool to a mucosal surface, wherein the endoscopic treatment tool includes a rod and, wherein the electrode protrudes from the rod in a radial direction of the rod;
passing an electric current through the electrode; and
coagulating the mucosal surface.

20. The treatment method according to claim 19, wherein abutting an electrode to the mucosal surface includes abutting a side surface of an insulator and the electrode to the mucosal surface, and wherein the endoscopic treatment tool includes an insulator on a distal end side of the rod.
